(19) United States
(12) Patent Application Publication
(10) Pub. No.: US 2022/0088155 A1
(43) Pub. Date: Mar. 24, 2022

(54) SUBCONJUNCTIVAL CHEMOTHERAPEUTIC DELIVERY BY FIBRIN SEALANT IN THE TREATMENT OF RETINOBLASTOMA

(71) Applicants: BAXTER INTERNATIONALINC., Deerfield, IL (US); BAXTER HEALTHCARE SA, Glattpark (Opfikon) (CH)

(72) Inventors: Christopher Ray HIMES, Chicago, IL (US); Timothy M. FULGHUM, Lakemoor, IL (US)

(21) Appl. No.: 17/419,509

(22) PCT Filed: Feb. 26, 2020
(86) PCT No.: PCT/US2020/019915

§ 371 Date: Jun. 29, 2021

Related U.S. Application Data
(60) Provisional application No. 62/810,681, filed on Feb. 26, 2019.
(60) Provisional application No. 62/925,639, filed on Oct. 24, 2019.
(60) Provisional application No. 62/943,916, filed on Dec. 5, 2019.

(51) Int. Cl. A61K 38/48; A61K 38/36; A61K 31/475; A61K 31/7048; A61K 31/282; A61K 31/198; A61K 31/704; A61K 31/513; A61K 31/17; A61K 31/519; A61P 35/00; A61M 5/19

(52) U.S. Cl. CPC A61K 38/4833; A61K 38/363; A61K 31/475; A61K 31/7048; A61K 31/282; A61K 31/198; A61M 2202/07; A61K 31/513; A61K 31/17; A61K 31/519; A61P 35/00; A61M 5/19; A61M 2202/064; A61K 31/704

(57) ABSTRACT

Methods and compositions for treating retinoblastoma are disclosed. The methods comprise administering a composition comprising two or more chemotherapeutic agents and a fibrin sealant material to a patient in need thereof. The compositions comprise two or more chemotherapeutic agents and a fibrin sealant material. Also disclosed are dual-compartment syringes comprising two or more chemotherapeutic agents, fibrinogen, and thrombin.

### CROSS-REFERENCES TO RELATED APPLICATIONS

This application claims the benefit of priority to U.S. Provisional Patent Application No. 62/810,681 filed Feb. 26, 2019, U.S. Provisional Patent Application No. 62/925,639 filed Oct. 24, 2019, and U.S. Provisional Patent Application No. 62/943,916 filed Dec. 5, 2019. The entire content of each of the aforementioned applications is incorporated herein by reference for all purposes.

### FIELD OF THE DISCLOSURE

The present invention relates to compositions and treatment methods for retinoblastoma.

### BACKGROUND

Retinoblastoma is the most common intraocular cancer in children, accounting for roughly 9,000 new cases every year and claiming the lives of up to 70% of cases in certain regions of the world. While modern retinoblastoma treatments in developed countries have significantly reduced mortality rates, post-treatment consequences such as vision loss, off-site organ dysfunction, and secondary metastases remain unmet clinical needs. The clinical standard of care consistently uses systemic administration of a chemotherapeutic cocktail of vincristine, etoposide, and carboplatin (VEC). Clinical trials have demonstrated that subconjunctival delivery of aqueous chemotherapy is an effective method of tumor suppression, likely due to significant permeability across the scleral membrane and diffusion through the vitreous. While high intraocular drug levels can be achieved through this approach, rapid clearance results in poor efficacy.

Fibrin sealants are FDA-approved biodegradable tissue adhesives that have been used clinically for hemostasis and tissue sealing. More recently they have been used within the contexts of cellular, gene, and chemotherapeutic delivery systems. Studies have explored the use of fibrin sealants to promote carboplatin retention at the scleral surface and to minimize the injections regimen necessary for tumor management. However, such studies did not explore a VEC combinatorial approach using fibrin sealants. There exists a need for more efficacious retinoblastoma treatments, particularly treatments that avoid rapid clearance.

### SUMMARY

The present disclosure provides compositions and methods for treating retinoblastoma. The compositions for treating retinoblastoma comprise two or more chemotherapeutic agents and a fibrin sealant material. The combination of two or more chemotherapeutic agents, such as the combination of vincristine, etoposide, and carboplatin (VEC), advantageously provides drug synergism.

The methods for treating retinoblastoma comprise administering a composition comprising two or more chemotherapeutic agents and a fibrin sealant material to a patient in need thereof.

The present disclosure also provides a dual-compartment syringe for administering the compositions disclosed herein according to the methods disclosed here. The dual-compartment syringe comprises two or more chemotherapeutic agents, fibrinogen, and thrombin.

An advantage of the drug matrix delivery system disclosed herein is that rapid drug clearance can be overcome.

A further advantage of the drug matrix delivery system disclosed herein is that a drug delivery system with fewer adverse effects compared with systemic delivery can be provided.

Yet another advantage of the disclosure is that fibrin polymerization (e.g., clot formation or structural attributes) is not appreciably affected by incorporation of etoposide and carboplatin in both whole blood and sealant formulations.

Still another advantage of the disclosure is that vincristine increases fibrin polymerization time without affecting overall clot strength (e.g., maximal clot amplitude or modulus).

An additional advantage of the disclosure is that compositional changes to the fibrin sealant can alter drug-matrix interactions, fluid permeability, and influence overall drug retention.

### BRIEF DESCRIPTION OF THE FIGURES

FIGS. 1A to 1F are graphs showing the inhibitory effect of cytotoxic compounds on Y79 and WERI retinoblastoma cells. Cell viability was assessed following a resazurin incubation and represented as a percentage to appropriate controls. Data are represented as mean+/−SD (n=5-8).

FIGS. 2A and 2B are graphs showing inhibitory effect of VEC combination treatments on Y79 and WERI retinoblastoma cells.

FIG. 3 depicts algorithms for computerized simulation of synergism, additivism and antagonism of the effect of multiple drugs.

FIG. 4A is a dose reduction plot generated for WERI cell lines (n=6).

FIG. 4B is a combination index plot generated for WERI cell lines (n=6).

FIG. 4C is a dose reduction plot generated for Y79 cell lines (n=6).

FIG. 4D is a combination index plot generated for Y79 cell lines (n=6).

FIG. 5A shows representative TEG curves and quantified outputs.

FIG. 5B shows a summary table of outputs normalized to the patient/sample control in whole blood (top) or fibrin sealant (bottom).

FIGS. 6A to 6D are graphs depicting thromboelastography (TEG) analysis of Chemotherapies in whole blood and fibrin sealant. Data are reported as fold changes from baseline controls ±SEM (n=5).

FIG. 6E depicts changes in TEG parameters reported as percent change from baseline ±SEM (n=5).

FIGS. 7A to 7F depict fibrin polymerization rates in blood plasma and fibrin sealant with VEC supplementation. Purified or reconstituted thrombin [5 IU/mL]/CaCl₂ [0.025M] were supplemented with chemotherapies VEC (125-1000 uM) prior to triggering clot formation in plasma or reconstituted fibrinogen (3 mg/mL) in 96-well plates. FIGS. 7A to 7C indicate fibrin formation rates over 40 minutes as determined 405 nm absorbance in blood plasma. FIGS. 7D to 7F indicate fibrin formation rates over 40 minutes as determined 405 nm absorbance in blood plasma fibrin sealants.

FIGS. 8A to 8D depict fiber analysis of VEC-embedded fibrin sealant. Commercially available fibrin sealant was admixed with 1 mM additions of Vincristine, Etoposide, or Carboplatin. Samples were critically point dried and imaged by scanning electron microscopy at 5,000× magnification at various points around the samples. Images were uploaded into ImageJ for fiber analysis. FIG. 8A shows a grayscale 2-D fibrin image. In FIG. 8B, the images were binarized to 8-bit grayscale. In FIG. 8C, the binarized image was skeletonized to produce a representation of the fiber network. The Analyze Skeleton function was used to quantify fiber branches and junctions. FIG. 8D shows a representative histogram output of branching numbers. Images analysed were taken at 5,000× magnificent and compared over a 800×800 pixel area.

FIG. 9 shows a plot of branch to junction ratios, where n=25-40 per treatment group. (*P<0.05).

FIG. 10 shows representative images of fiber structures by treatment group.

FIG. 11 illustrates a representative embodiment of a dual-compartment syringe.

### DETAILED DESCRIPTION

The present disclosure provides methods and compositions for treating retinoblastoma. The present disclosure also provides a dual-compartment syringe for delivering the disclosed compositions according to the disclosed methods.

### Methods for Treating Retinoblastoma

In an embodiment of the present disclosure, which may be combined with any other embodiment listed herein unless specified otherwise, a method for treating retinoblastoma is provided comprising: administering a composition comprising two or more chemotherapeutic agents and a fibrin sealant material to a patient in need thereof.

In an embodiment of the present disclosure, which may be combined with any other embodiment listed herein unless specified otherwise, the chemotherapeutic agents are selected from the group consisting of: vincristine, etoposide, carboplatin, melphalan, doxorubicin, fluorouracil, carmustine, methotrexate, and mixtures thereof.

In an embodiment of the present disclosure, which may be combined with any other embodiment listed herein unless specified otherwise, the chemotherapeutic agents are a mixture of vincristine, etoposide, and carboplatin.

In an embodiment of the present disclosure, which may be combined with any other embodiment listed herein unless specified otherwise, each chemotherapeutic agent is independently present in the composition in a concentration of about 1 uM to about 1 mM, such as about 10 uM to about 1 mM, about 50 uM to about 500 uM, about 100 uM to about 250 uM, about 100 uM to about 200 uM, about 100 uM to about 150 uM, about 1 uM to about 5 uM, about 5 uM to about 10 uM, about 10 uM to about 20 uM, about 20 uM to about 30 uM, about 30 uM to about 40 uM, about 40 uM to about 50 uM, about 50 uM to about 60 uM, about 60 uM to about 70 uM, about 70 uM to about 80 uM, about 80 uM to about 90 uM, about 90 uM to about 100 uM, about 100 uM to about 120 uM, about 120 uM to about 140 uM, about 140 uM to about 160 uM, about 160 uM to about 180 uM, about 180 uM to about 200 uM, about 200 uM to about 220 uM, about 220 uM to about 240 uM, about 240 uM to about 260 uM, about 260 uM to about 280 uM, about 280 uM to about 300 uM, about 300 uM to about 350 uM, about 350 uM to about 400 uM, about 400 uM to about 500 uM, about 500 uM to about 600 uM, about 600 uM to about 700 uM, about 700 uM to about 800 uM, about 800 uM to about 900 uM, and/or about 900 uM to about 1 mM.

In an embodiment of the present disclosure, which may be combined with any other embodiment listed herein unless specified otherwise, the chemotherapeutic agents are present in the composition in a total concentration of about 3 um to about 3 mM, such as about 30 uM to about 3 mM, about 150 uM to about 1.5 mM, about 300 uM to about 750 uM, about 300 uM to about 600 uM, about 300 uM to about 450 uM, about 3 uM to about 15 uM, about 15 uM to about 30 uM, about 30 uM to about 60 uM, about 60 uM to about 90 uM, about 90 uM to about 120 uM, about 120 uM to about 150 uM, about 150 uM to about 180 uM, about 180 uM to about 210 uM, about 210 uM to about 240 uM, about 240 uM to about 270 uM, about 270 uM to about 300 uM, about 300 uM to about 360 uM, about 360 uM to about 420 uM, about 420 uM to about 480 uM, about 480 uM to about 540 uM, about 540 uM to about 600 uM, about 600 uM to about 660 uM, about 660 uM to about 720 uM, about 720 uM to about 780 uM, about 780 uM to about 840 uM, about 840 uM to about 900 uM, about 900 uM to about 1050 uM, about 1050 uM to about 1200 uM, about 1200 uM to about 1.5 mM, about 1.5 mM to about 1.8 mM, about 1.8 mM to about 2.1 mM, about 2.1 mM to about 2.4 mM, about 2.4 mM to about 2.7 mM, and/or about 2.7 to about 3 mM.

In an embodiment of the present disclosure, which may be combined with any other embodiment listed herein unless specified otherwise, the chemotherapeutic agents are a mixture of vincristine, etoposide, and carboplatin, and the vincristine is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%. In an embodiment of the present disclosure, which may be combined with any other embodiment listed herein unless specified otherwise, the chemotherapeutic agents are a mixture of vincristine, etoposide, and carboplatin, and the vincristine is present in an amount of about 0.05% to about 10% of the chemotherapeutical agents on a molar basis, such as about 0.05% to about 0.1%, about 0.1% to about 0.2%, about 0.2% to about 0.3%, about 0.3% to about 0.4%, about 0.4% to about 0.5%, about 0.5% to about 1%, about 1% to about 2%, about 2% to about 5%, and/or about 5% to about 10%.

In an embodiment of the present disclosure, which may be combined with any other embodiment listed herein unless specified otherwise, the chemotherapeutic agents are a mixture of vincristine, etoposide, and carboplatin, and the etoposide is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

In an embodiment of the present disclosure, which may be combined with any other embodiment listed herein unless specified otherwise, the chemotherapeutic agents are a mixture of vincristine, etoposide, and carboplatin, and the carboplatin is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.05% to about 10% vincristine, about 10% to about 80% etoposide, and about 10% to about 80% carboplatin on a molar basis of the chemotherapeutical agents, such as about 0.1% to about 5% vincristine, about 15% to about 60% etoposide, and about 40% to about 80% carboplatin, about 0.1% to about 1% vincristine, about 20% to about 50% etoposide, and about 50% to about 80% carboplatin, about 0.1% to about 0.5% vincristine, about 25% to about 45% etoposide, and about 55% to about 75% carboplatin, about 0.1% to about 0.2% vincristine, about 30% to about 40% etoposide, and about 60% to about 70% carboplatin, and/or about 0.2% vincristine, about 34.9% etoposide, and about 64.9% carboplatin. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.7% vincristine, about 33.1% etoposide, and about 66.2% carboplatin on a molar basis of the chemotherapeutical agents. In some cases, these chemotherapeutic mixtures are used treating for non-metastatic type retinoblastomas. For example, in some cases, a chemotherapeutic mixture of vincristine, etoposide, and carboplatin in a molar ratio of about 1:50:100 is used for treating non-metastatic type retinoblastomas. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.02% to about 1% vincristine, about 30% to about 90% etoposide, and about 10% to about 70% carboplatin on a molar basis of the chemotherapeutical agents, such as about 0.02% to about 0.5% vincristine, about 40% to about 80% etoposide, and about 20% to about 60% carboplatin, about 0.02% to about 0.1% vincristine, about 50% to about 70% etoposide, and about 25% to about 50% carboplatin, about 0.02% to about 0.05% vincristine, about 50% to about 70% etoposide, and about 25% to about 45% carboplatin, and/or about 0.03% to about 0.04% vincristine, about 60% to about 70% etoposide, and about 30% to about 40% carboplatin. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.033% vincristine, about 66.644% etoposide, and about 33.322% carboplatin on a molar basis of the chemotherapeutical agents. In some cases, these chemotherapeutic mixtures are used treating for metastatically-prone retinoblastomas. For example, in some cases, a chemotherapeutic mixture of vincristine, etoposide, and carboplatin in a molar ratio of about 1:2000:1000 is used for treating metastatically-prone retinoblastomas.

In an embodiment of the present disclosure, which may be combined with any other embodiment listed herein unless specified otherwise, the fibrin sealant material comprises fibrinogen and thrombin.

In an embodiment of the present disclosure, which may be combined with any other embodiment listed herein unless specified otherwise, the fibrin sealant material comprises fibrinogen in a concentration of about 10 mg/mL to about 200 mg/mL, such as about 30 mg/mL to about 150 mg/mL, about 50 mg/mL to about 120 mg/mL, about 75 mg/mL to about 125 mg/mL, about 80 mg/mL to about 100 mg/mL, about 90 mg/mL, about 10 mg/mL to about 30 mg/mL, about 30 mg/mL to about 50 mg/mL, about 50 mg/mL to about 70 mg/mL, about 70 mg/mL to about 90 mg/mL, about 90 mg/mL to about 110 mg/mL, about 110 mg/mL to about 130 mg/mL, about 130 mg/mL to about 150 mg/mL, about 150 mg/mL to about 170 mg/mL, and/or about 170 mg/mL to about 200 mg/mL.

In an embodiment of the present disclosure, which may be combined with any other embodiment listed herein unless specified otherwise, the fibrin sealant material comprises thrombin in a concentration of about 10 IU/mL to about 2000 IU/mL, such as about 50 IU/mL to about 1000 IU/mL, about 100 IU/mL to about 800 IU/mL, about 250 IU/mL to about 750 IU/mL, about 300 IU/mL to about 700 IU/mL, about 400 IU/mL to about 600 IU/mL, and/or about 500 IU/mL.

In an embodiment of the present disclosure, which may be combined with any other embodiment listed herein unless specified otherwise, the composition is administered using a dual-compartment syringe.

In an embodiment of the present disclosure, which may be combined with any other embodiment listed herein unless specified otherwise, a first compartment of the syringe comprises the chemotherapeutic agents and thrombin, and a second compartment of the syringe comprises fibrinogen.

In an embodiment of the present disclosure, which may be combined with any other embodiment listed herein unless specified otherwise, a first compartment of the syringe comprises the chemotherapeutic agents and fibrinogen, and a second compartment of the syringe comprises thrombin.

In an embodiment of the present disclosure, which may be combined with any other embodiment listed herein unless specified otherwise, the composition is administered by subconjunctival injection.

### Compositions for Treating Retinoblastoma

In an embodiment of the present disclosure, which may be combined with any other embodiment listed herein unless specified otherwise, a composition for treating retinoblastoma is provided comprising: two or more chemotherapeutic agents and a fibrin sealant material.

In an embodiment of the present disclosure, which may be combined with any other embodiment listed herein unless specified otherwise, the chemotherapeutic agents are selected from the group consisting of: vincristine, etoposide, carboplatin, melphalan, doxorubicin, fluorouracil, carmustine, methotrexate, and mixtures thereof.

In an embodiment of the present disclosure, which may be combined with any other embodiment listed herein unless specified otherwise, the chemotherapeutic agents are a mixture of vincristine, etoposide, and carboplatin.

In an embodiment of the present disclosure, which may be combined with any other embodiment listed herein unless specified otherwise, each chemotherapeutic agent is independently present in the composition in a concentration of about 1 uM to about 1 mM, such as about 10 uM to about 1 mM, about 50 uM to about 500 uM, about 100 uM to about 250 uM, about 100 uM to about 200 uM, about 100 uM to about 150 uM, about 1 uM to about 5 uM, about 5 uM to about 10 uM, about 10 uM to about 20 uM, about 20 uM to about 30 uM, about 30 uM to about 40 uM, about 40 uM to about 50 uM, about 50 uM to about 60 uM, about 60 uM to about 70 uM, about 70 uM to about 80 uM, about 80 uM to about 90 uM, about 90 uM to about 100 uM, about 100 uM to about 120 uM, about 120 uM to about 140 uM, about 140 uM to about 160 uM, about 160 uM to about 180 uM, about 180 uM to about 200 uM, about 200 uM to about 220 uM, about 220 uM to about 240 uM, about 240 uM to about 260 uM, about 260 uM to about 280 uM, about 280 uM to about 300 uM, about 300 uM to about 350 uM, about 350 uM to about 400 uM, about 400 uM to about 500 uM, about 500 uM to about 600 uM, about 600 uM to about 700 uM, about 700 uM to about 800 uM, about 800 uM to about 900 uM, and/or about 900 uM to about 1 mM.

In an embodiment of the present disclosure, which may be combined with any other embodiment listed herein unless specified otherwise, the chemotherapeutic agents are present in the composition in a total concentration of about 3 um to about 3 mM, such as about 30 uM to about 3 mM, about 150 uM to about 1.5 mM, about 300 uM to about 750 uM, about 300 uM to about 600 uM, about 300 uM to about 450 uM, about 3 uM to about 15 uM, about 15 uM to about 30 uM, about 30 uM to about 60 uM, about 60 uM to about 90 uM, about 90 uM to about 120 uM, about 120 uM to about 150 uM, about 150 uM to about 180 uM, about 180 uM to about 210 uM, about 210 uM to about 240 uM, about 240 uM to about 270 uM, about 270 uM to about 300 uM, about 300 uM to about 360 uM, about 360 uM to about 420 uM, about 420 uM to about 480 uM, about 480 uM to about 540 uM, about 540 uM to about 600 uM, about 600 uM to about 660 uM, about 660 uM to about 720 uM, about 720 uM to about 780 uM, about 780 uM to about 840 uM, about 840 uM to about 900 uM, about 900 uM to about 1050 uM, about 1050 uM to about 1200 uM, about 1200 uM to about 1.5 mM, about 1.5 mM to about 1.8 mM, about 1.8 mM to about 2.1 mM, about 2.1 mM to about 2.4 mM, about 2.4 mM to about 2.7 mM, and/or about 2.7 to about 3 mM.

In an embodiment of the present disclosure, which may be combined with any other embodiment listed herein unless specified otherwise, the chemotherapeutic agents are a mixture of vincristine, etoposide, and carboplatin, and the vincristine is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%. In an embodiment of the present disclosure, which may be combined with any other embodiment listed herein unless specified otherwise, the chemotherapeutic agents are a mixture of vincristine, etoposide, and carboplatin, and the vincristine is present in an amount of about 0.05% to about 10% of the chemotherapeutical agents on a molar basis, such as about 0.05% to about 0.1%, about 0.1% to about 0.2%, about 0.2% to about 0.3%, about 0.3% to about 0.4%, about 0.4% to about 0.5%, about 0.5% to about 1%, about 1% to about 2%, about 2% to about 5%, and/or about 5% to about 10%.

In an embodiment of the present disclosure, which may be combined with any other embodiment listed herein unless specified otherwise, the chemotherapeutic agents are a mixture of vincristine, etoposide, and carboplatin, and the etoposide is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

In an embodiment of the present disclosure, which may be combined with any other embodiment listed herein unless specified otherwise, the chemotherapeutic agents are a mixture of vincristine, etoposide, and carboplatin, and the carboplatin is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.05% to about 10% vincristine, about 10% to about 80% etoposide, and about 10% to about 80% carboplatin on a molar basis of the chemotherapeutical agents, such as about 0.1% to about 5% vincristine, about 15% to about 60% etoposide, and about 40% to about 80% carboplatin, about 0.1% to about 1% vincristine, about 20% to about 50% etoposide, and about 50% to about 80% carboplatin, about 0.1% to about 0.5% vincristine, about 25% to about 45% etoposide, and about 55% to about 75% carboplatin, about 0.1% to about 0.2% vincristine, about 30% to about 40% etoposide, and about 60% to about 70% carboplatin, and/or about 0.2% vincristine, about 34.9% etoposide, and about 64.9% carboplatin. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.7% vincristine, about 33.1% etoposide, and about 66.2% carboplatin on a molar basis of the chemotherapeutical agents. In some cases, these chemotherapeutic mixtures are used treating for non-metastatic type retinoblastomas. For example, in some cases, a chemotherapeutic mixture of vincristine, etoposide, and carboplatin in a molar ratio of about 1:50:100 is used for treating non-metastatic type retinoblastomas. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.02% to about 1% vincristine, about 30% to about 90% etoposide, and about 10% to about 70% carboplatin on a molar basis of the chemotherapeutical agents, such as about 0.02% to about 0.5% vincristine, about 40% to about 80% etoposide, and about 20% to about 60% carboplatin, about 0.02% to about 0.1% vincristine, about 50% to about 70% etoposide, and about 25% to about 50% carboplatin, about 0.02% to about 0.05% vincristine, about 50% to about 70% etoposide, and about 25% to about 45% carboplatin, and/or about 0.03% to about 0.04% vincristine, about 60% to about 70% etoposide, and about 30% to about 40% carboplatin. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.033% vincristine, about 66.644% etoposide, and about 33.322% carboplatin on a molar basis of the chemotherapeutical agents. In some cases, these chemotherapeutic mixtures are used treating for metastatically-prone retinoblastomas. For example, in some cases, a chemotherapeutic mixture of vincristine, etoposide, and carboplatin in a molar ratio of about 1:2000:1000 is used for treating metastatically-prone retinoblastomas.

In an embodiment of the present disclosure, which may be combined with any other embodiment listed herein unless specified otherwise, the fibrin sealant material comprises fibrinogen and thrombin.

In an embodiment of the present disclosure, which may be combined with any other embodiment listed herein unless specified otherwise, the fibrin sealant material comprises fibrinogen in a concentration of about 10 mg/mL to about 200 mg/mL, such as about 30 mg/mL to about 150 mg/mL, about 50 mg/mL to about 120 mg/mL, about 75 mg/mL to about 125 mg/mL, about 80 mg/mL to about 100 mg/mL, about 90 mg/mL, about 10 mg/mL to about 30 mg/mL, about 30 mg/mL to about 50 mg/mL, about 50 mg/mL to about 70 mg/mL, about 70 mg/mL to about 90 mg/mL, about 90 mg/mL to about 110 mg/mL, about 110 mg/mL to about 130 mg/mL, about 130 mg/mL to about 150 mg/mL, about 150 mg/mL to about 170 mg/mL, and/or about 170 mg/mL to about 200 mg/mL.

In an embodiment of the present disclosure, which may be combined with any other embodiment listed herein unless specified otherwise, the fibrin sealant material comprises thrombin in a concentration of about 10 IU/mL to about 2000 IU/mL, such as about 50 IU/mL to about 1000 IU/mL, about 100 IU/mL to about 800 IU/mL, about 250 IU/mL to about 750 IU/mL, about 300 IU/mL to about 700 IU/mL, about 400 IU/mL to about 600 IU/mL, and/or about 500 IU/mL.

In an embodiment of the present disclosure, which may be combined with any other embodiment listed herein unless specified otherwise, the fibrin sealant material is obtained by combining a first solution comprising the chemotherapeutical agents and thrombin with a second solution comprising fibrinogen.

In an embodiment of the present disclosure, which may be combined with any other embodiment listed herein unless specified otherwise, the fibrin sealant material is obtained by combining a first solution comprising fibrinogen with a second solution comprising the chemotherapeutical agents and thrombin.

### Dual-Compartment Syringes

In an embodiment of the present disclosure, which may be combined with any other embodiment listed herein unless specified otherwise, a dual-compartment syringe is provided comprising two or more chemotherapeutic agents, fibrinogen, and thrombin.

In an embodiment of the present disclosure, which may be combined with any other embodiment listed herein unless specified otherwise, a first compartment of the syringe comprises the chemotherapeutic agents and thrombin, and a second compartment of the syringe comprises fibrinogen.

In an embodiment of the present disclosure, which may be combined with any other embodiment listed herein unless specified otherwise, the chemotherapeutic agents and thrombin are present as a solution, and the fibrinogen is present as a solution.

In an embodiment of the present disclosure, which may be combined with any other embodiment listed herein unless specified otherwise, the solutions are frozen.

In an embodiment of the present disclosure, which may be combined with any other embodiment listed herein unless specified otherwise, the chemotherapeutic agents and thrombin are present as a dry powder, and the fibrinogen is present as a dry powder.

In an embodiment of the present disclosure, which may be combined with any other embodiment listed herein unless specified otherwise, the dry powders are reconstituted to form a solution.

In an embodiment of the present disclosure, which may be combined with any other embodiment listed herein unless specified otherwise, a first compartment of the syringe comprises the chemotherapeutic agents and fibrinogen, and a second compartment of the syringe comprises thrombin.

In an embodiment of the present disclosure, which may be combined with any other embodiment listed herein unless specified otherwise, the chemotherapeutic agents are selected from the group consisting of: vincristine, etoposide, carboplatin, melphalan, doxorubicin, fluorouracil, carmustine, methotrexate, and mixtures thereof.

In an embodiment of the present disclosure, which may be combined with any other embodiment listed herein unless specified otherwise, the chemotherapeutic agents are a mixture of vincristine, etoposide, and carboplatin.

In an embodiment of the present disclosure, which may be combined with any other embodiment listed herein unless specified otherwise, each chemotherapeutic agent is independently present in the composition in a concentration of about 1 uM to about 1 mM, such as about 10 uM to about 1 mM, about 50 uM to about 500 uM, about 100 uM to about 250 uM, about 100 uM to about 200 uM, about 100 uM to about 150 uM, about 1 uM to about 5 uM, about 5 uM to about 10 uM, about 10 uM to about 20 uM, about 20 uM to about 30 uM, about 30 uM to about 40 uM, about 40 uM to about 50 uM, about 50 uM to about 60 uM, about 60 uM to about 70 uM, about 70 uM to about 80 uM, about 80 uM to about 90 uM, about 90 uM to about 100 uM, about 100 uM to about 120 uM, about 120 uM to about 140 uM, about 140 uM to about 160 uM, about 160 uM to about 180 uM, about 180 uM to about 200 uM, about 200 uM to about 220 uM, about 220 uM to about 240 uM, about 240 uM to about 260 uM, about 260 uM to about 280 uM, about 280 uM to about 300 uM, about 300 uM to about 350 uM, about 350 uM to about 400 uM, about 400 uM to about 500 uM, about 500 uM to about 600 uM, about 600 uM to about 700 uM, about 700 uM to about 800 uM, about 800 uM to about 900 uM, and/or about 900 uM to about 1 mM.

In an embodiment of the present disclosure, which may be combined with any other embodiment listed herein unless specified otherwise, the chemotherapeutic agents are present in the composition in a total concentration of about 3 um to about 3 mM, such as about 30 uM to about 3 mM, about 150 uM to about 1.5 mM, about 300 uM to about 750 uM, about 300 uM to about 600 uM, about 300 uM to about 450 uM, about 3 uM to about 15 uM, about 15 uM to about 30 uM, about 30 uM to about 60 uM, about 60 uM to about 90 uM, about 90 uM to about 120 uM, about 120 uM to about 150 uM, about 150 uM to about 180 uM, about 180 uM to about 210 uM, about 210 uM to about 240 uM, about 240 uM to about 270 uM, about 270 uM to about 300 uM, about 300 uM to about 360 uM, about 360 uM to about 420 uM, about 420 uM to about 480 uM, about 480 uM to about 540 uM, about 540 uM to about 600 uM, about 600 uM to about 660 uM, about 660 uM to about 720 uM, about 720 uM to about 780 uM, about 780 uM to about 840 uM, about 840 uM to about 900 uM, about 900 uM to about 1050 uM, about 1050 uM to about 1200 uM, about 1200 uM to about 1.5 mM, about 1.5 mM to about 1.8 mM, about 1.8 mM to about 2.1 mM, about 2.1 mM to about 2.4 mM, about 2.4 mM to about 2.7 mM, and/or about 2.7 to about 3 mM.

In an embodiment of the present disclosure, which may be combined with any other embodiment listed herein unless specified otherwise, the chemotherapeutic agents are a mixture of vincristine, etoposide, and carboplatin, and the vincristine is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%. In an embodiment of the present disclosure, which may be combined with any other embodiment listed herein unless specified otherwise, the chemotherapeutic agents are a mixture of vincristine, etoposide, and carboplatin, and the vincristine is present in an amount of about 0.05% to about 10% of the chemotherapeutical agents on a molar basis, such as about 0.05% to about 0.1%, about 0.1% to about 0.2%, about 0.2% to about 0.3%, about 0.3% to about 0.4%, about 0.4% to about 0.5%, about 0.5% to about 1%, about 1% to about 2%, about 2% to about 5%, and/or about 5% to about 10%.

In an embodiment of the present disclosure, which may be combined with any other embodiment listed herein unless specified otherwise, the chemotherapeutic agents are a mixture of vincristine, etoposide, and carboplatin, and the etoposide is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

In an embodiment of the present disclosure, which may be combined with any other embodiment listed herein unless specified otherwise, the chemotherapeutic agents are a mixture of vincristine, etoposide, and carboplatin, and the carboplatin is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.05% to about 10% vincristine, about 10% to about 80% etoposide, and about 10% to about 80% carboplatin on a molar basis of the chemotherapeutical agents, such as about 0.1% to about 5% vincristine, about 15% to about 60% etoposide, and about 40% to about 80% carboplatin, about 0.1% to about 1% vincristine, about 20% to about 50% etoposide, and about 50% to about 80% carboplatin, about 0.1% to about 0.5% vincristine, about 25% to about 45% etoposide, and about 55% to about 75% carboplatin, about 0.1% to about 0.2% vincristine, about 30% to about 40% etoposide, and about 60% to about 70% carboplatin, and/or about 0.2% vincristine, about 34.9% etoposide, and about 64.9% carboplatin. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.7% vincristine, about 33.1% etoposide, and about 66.2% carboplatin on a molar basis of the chemotherapeutical agents. In some cases, these chemotherapeutic mixtures are used treating for non-metastatic type retinoblastomas. For example, in some cases, a chemotherapeutic mixture of vincristine, etoposide, and carboplatin in a molar ratio of about 1:50:100 is used for treating non-metastatic type retinoblastomas. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.02% to about 1% vincristine, about 30% to about 90% etoposide, and about 10% to about 70% carboplatin on a molar basis of the chemotherapeutical agents, such as about 0.02% to about 0.5% vincristine, about 40% to about 80% etoposide, and about 20% to about 60% carboplatin, about 0.02% to about 0.1% vincristine, about 50% to about 70% etoposide, and about 25% to about 50% carboplatin, about 0.02% to about 0.05% vincristine, about 50% to about 70% etoposide, and about 25% to about 45% carboplatin, and/or about 0.03% to about 0.04% vincristine, about 60% to about 70% etoposide, and about 30% to about 40% carboplatin. In an embodiment of the present disclosure, which may be combined with any other embodiment listed herein unless specified otherwise, the chemotherapeutic agents are a mixture of vincristine, etoposide, and carboplatin, and the chemotherapeutic agents are present in an amount of about 0.033% vincristine, about 66.644% etoposide, and about 33.322% carboplatin on a molar basis of the chemotherapeutical agents. In some cases, these chemotherapeutic mixtures are used treating for metastatically-prone retinoblastomas. For example, in some cases, a chemotherapeutic mixture of vincristine, etoposide, and carboplatin in a molar ratio of about 1:2000:1000 is used for treating metastatically-prone retinoblastomas.

In an embodiment of the present disclosure, which may be combined with any other embodiment listed herein unless specified otherwise, the fibrinogen is present in a concentration of about 10 mg/mL to about 200 mg/mL, such as about 30 mg/mL to about 150 mg/mL, about 50 mg/mL to about 120 mg/mL, about 75 mg/mL to about 125 mg/mL, about 80 mg/mL to about 100 mg/mL, about 90 mg/mL, about 10 mg/mL to about 30 mg/mL, about 30 mg/mL to about 50 mg/mL, about 50 mg/mL to about 70 mg/mL, about 70 mg/mL to about 90 mg/mL, about 90 mg/mL to about 110 mg/mL, about 110 mg/mL to about 130 mg/mL, about 130 mg/mL to about 150 mg/mL, about 150 mg/mL to about 170 mg/mL, and/or about 170 mg/mL to about 200 mg/mL.

In an embodiment of the present disclosure, which may be combined with any other embodiment listed herein unless specified otherwise, the thrombin is present in a concentration of about 10 IU/mL to about 2000 IU/mL, such as about 50 IU/mL to about 1000 IU/mL, about 100 IU/mL to about 800 IU/mL, about 250 IU/mL to about 750 IU/mL, about 300 IU/mL to about 700 IU/mL, about 400 IU/mL to about 600 IU/mL, and/or about 500 IU/mL.

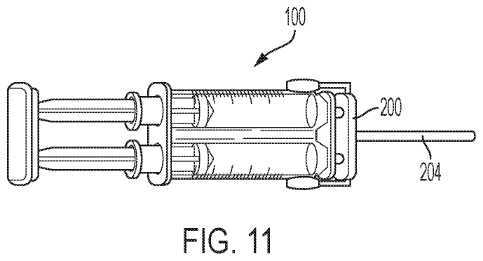

As illustrated in FIG. 11, dual-compartment syringe 100 includes an applicator tip having a Y-connector 200 and a dispensing needle 204 that is coupled to and projects outward from the Y-connector 200.

The invention is further described in the examples below, yet without being restricted thereto.

### EXAMPLES

### Example 1

### Methods

Cell Culture

The human retinoblastoma Y79 (ATCC® HTB18™) and WERI (ATCC® HTB169™) cell lines were cultured in RPMI 1640 (ATCC®) medium supplemented with 20% (Y79) or 10% (WERI-RB) fetal bovine serum, 100 U/mL penicillin, and 100 mg/mL streptomycin in a humidified atmosphere of 5% CO₂ at 37° C., grown in 75 cm² tissue culture flasks. Once confluent, cells were sub cultivated with fresh medium 1:2 to 1:4 ratios after discarding the supernatant and obtaining the gravity-settled aggregates.

Cell Viability Assays

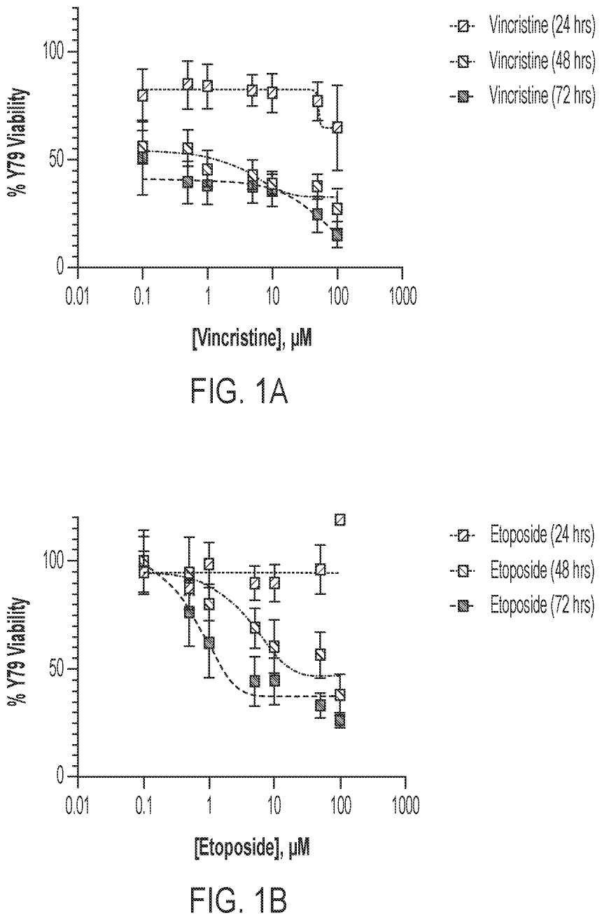

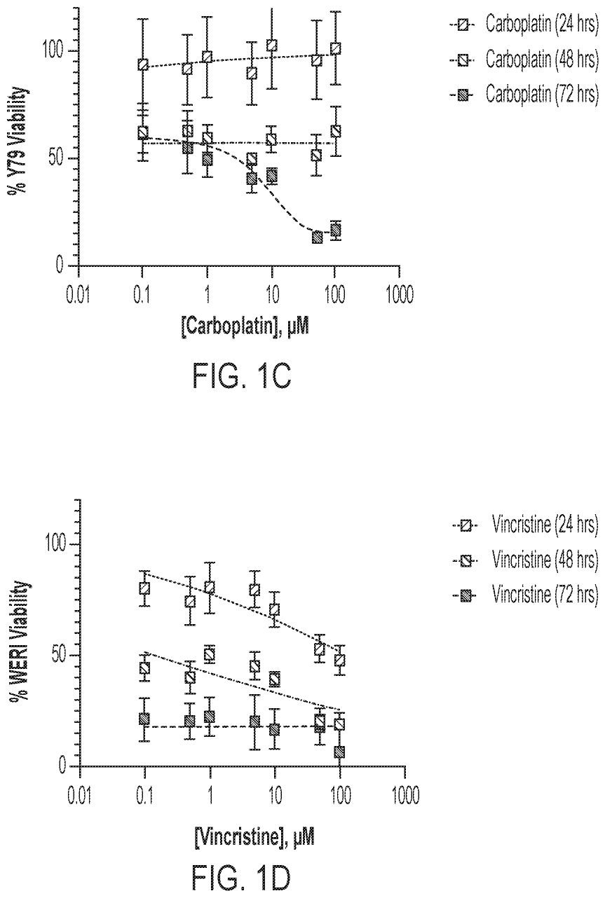

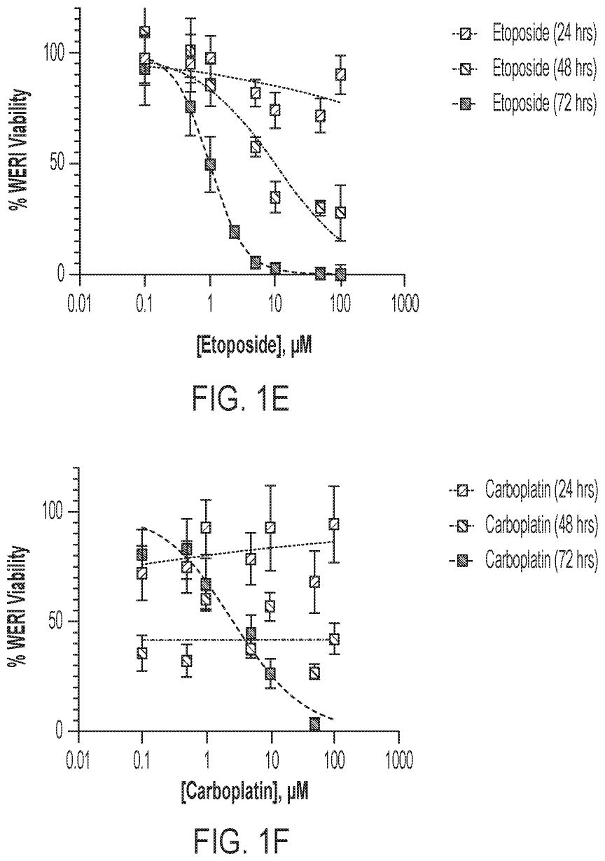

10 mL of cell suspension were centrifuged at 1000 G for 10 minutes. The supernatant was discarded and the pellet was then resuspended in media to 1.1*10\hat{ }5 live cells/mL following a viability cell count check. Cells were plated in 96-well plates at 10,000 cells per well. Sample wells were treated with carboplatin, etoposide or vincristine at the concentrations of 1000 uM, 100 uM, 50 uM, 10 uM, 5 uM, 1 uM, 0.5 uM, and 0.1 uM. Untreated and non-cell controls include a 10 uL vehicle, bringing up all wells to 100 uL total volume. Experiments covered 24/48/72 hour incubations at 5% CO₂ at 37° C. prior to analyses. Plates ready for analysis were incubated with 10 uL of 10× Alamar Blue Reagent for 1-4 hours (apparent color change) and emission intensity read at 585 nm following a 555 nm excitation. The results are shown in FIG. 1.

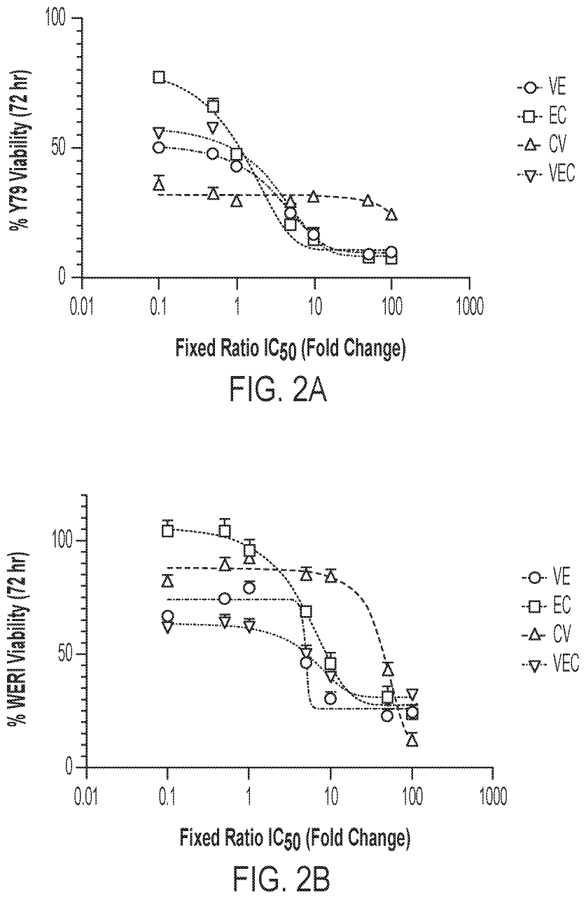

Y79 and WERI cells were incubated with Vincristine, Etoposide, or Carboplatin using previously established half-maximal inhibitory concentrations in a fixed-ratio treatment paradigm. Y79 and WERI cells also were incubated with a mixture of Vincristine, Etoposide, or Carboplatin. Cell viability was assessed following a resazurin incubation and represented as a percentage to appropriate Y79 and WERI controls, as shown in FIGS. 2A and 2B, respectively.

Combination Analyses

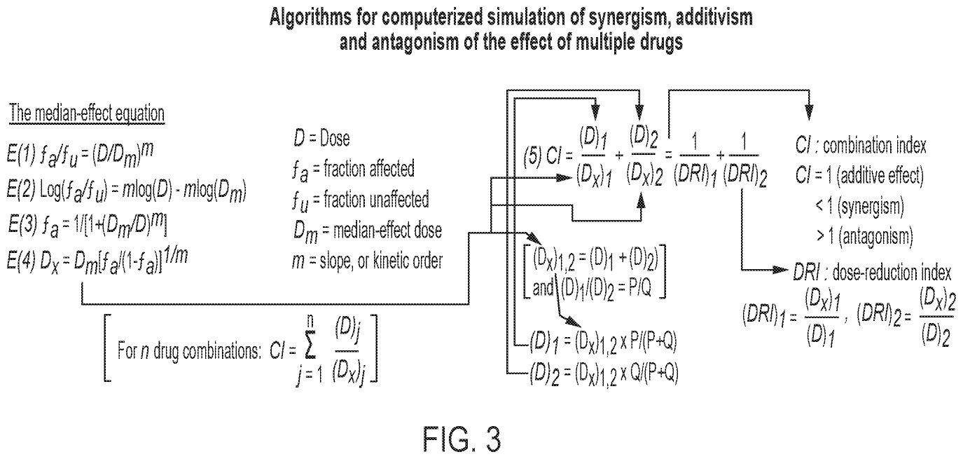

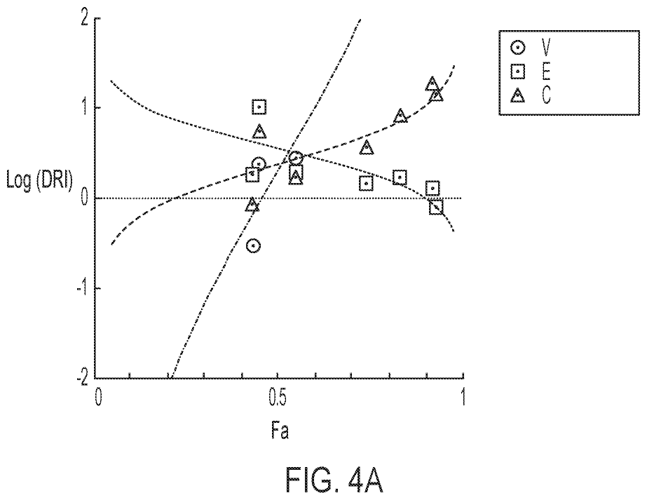

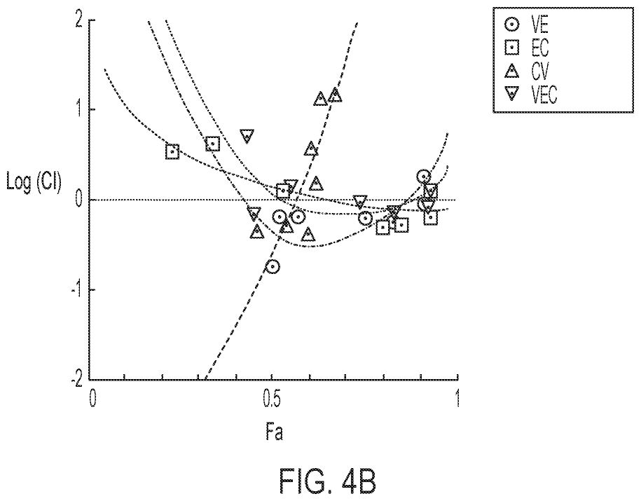

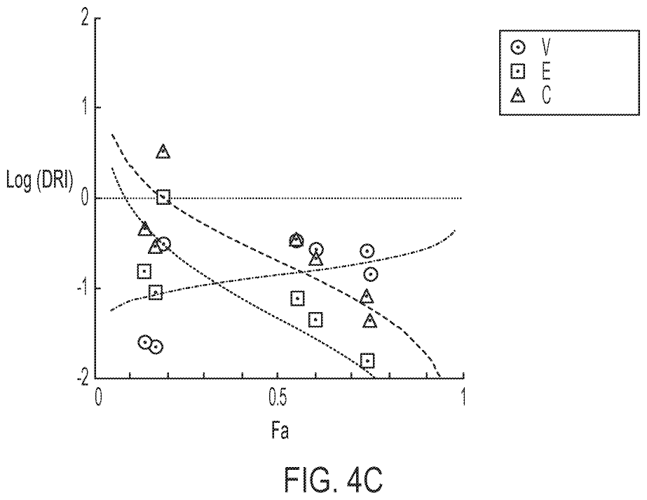

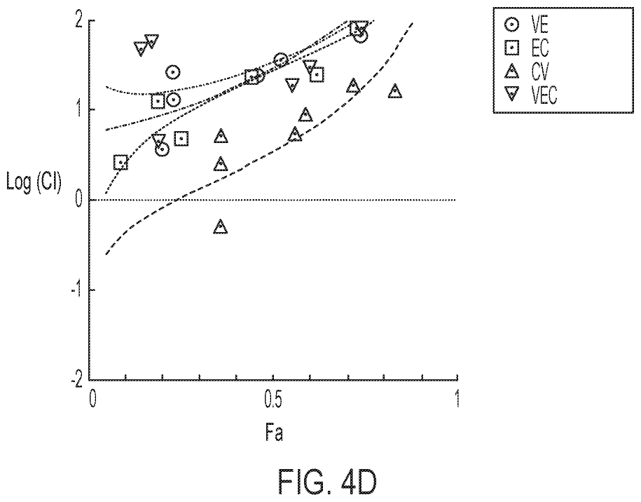

The combination effects of VEC were analyzed using combination index (CI) and dose reduction index (DRI) analyses. Plots were generated using CompuSyn software employing the Chou-Talalay calculation methods of determining drug synergism as depicted in FIG. 3. Representative plots are shown in FIGS. 4A to 4D.

Transwell Viability Assays

Fibrin sealant molds (0.5 mL) were prepared in 12 mm transwell inserts using 5 IU/mL thrombin admixed with fixed molar VEC ratios previously estimated upon the half maximal inhibitory concentrations of Y79 and WERI cell lines. Ratios were tested at 0.1, 0.5, 1, 50, and 100-fold concentrations. The discs were incubated for 72 hours in 1.5 mL full growth media with Y79 or WERI (2.2*10\hat{ }5 live cells/mL). After 72 hours, wells were incubated with Alamar Blue Reagent for 1-4 hours (apparent color change) and emission intensity read at 585 nm following a 555 nm excitation. Dose-response curves for each drug were generated and the inhibitory concentration values extrapolated.

Thromboelastography Analysis of Chemotherapies in Whole Blood and Fibrin Glue

Alterations in the clot strength and formation kinetics were determined in whole blood and fibrin sealant following additions of Vincristine, Etoposide, or Carboplatin.

Clot formation kinetics and strength were determined with thromboelastography (TEG) using Thromboelastograph® Hemostasis Analyzer Model 5000 (Haemonetics Co.) with the following deviations. The initial set of experiments were prepared according to standard TEG operating procedures, testing for alterations in the clotting properties following additions of chemotherapeutic Vincristine, Etoposide, and Carboplatin compared to a saline or DMSO controls. In short, fifty-six (56 uL) of a CaCl₂ [0.011M]/chemotherapy [1 mM] solution were placed in cups wells prior to the addition of 304 uL of whole blood for a total well volume of 360 uL. The TEG analyses were started at t=15 seconds for all sets of samples and run at 37° C.

An additional set of experiments were performed using TISSEEL™ fibrin glue, using reconstituted fibrinogen and thrombin mixtures to generate a clot. To begin, 304 uL of reconstituted fibrinogen protein concentrate was diluted to physiological plasma concentrations (˜3 mg/mL) and placed in the cup wells. Fifty-six (56 uL) of reconstituted thrombin [5 IU/mL]/chemotherapy [1 mM] were then added, bringing the total well volume of 360 uL. The TEG analyses were started at t=15 seconds for all sets of samples and run at 37° C.

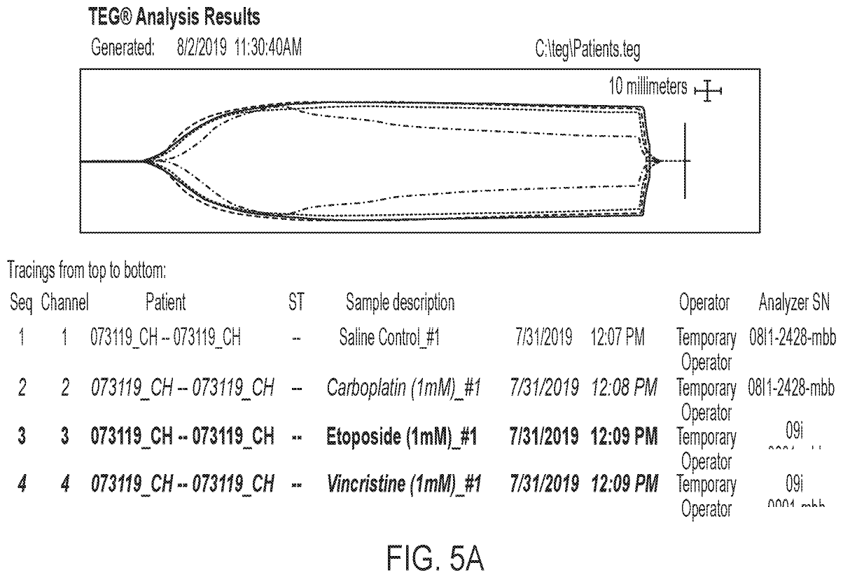

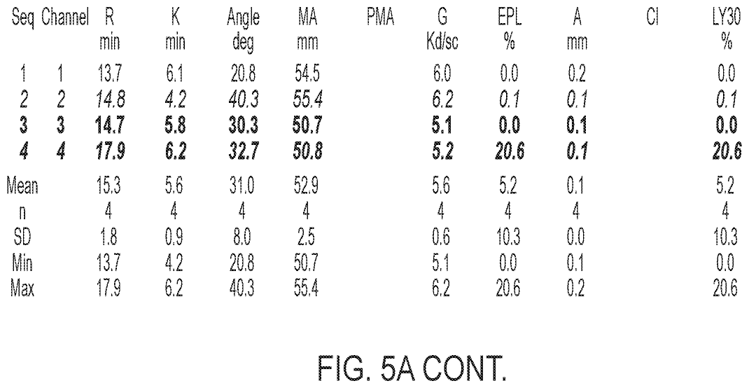

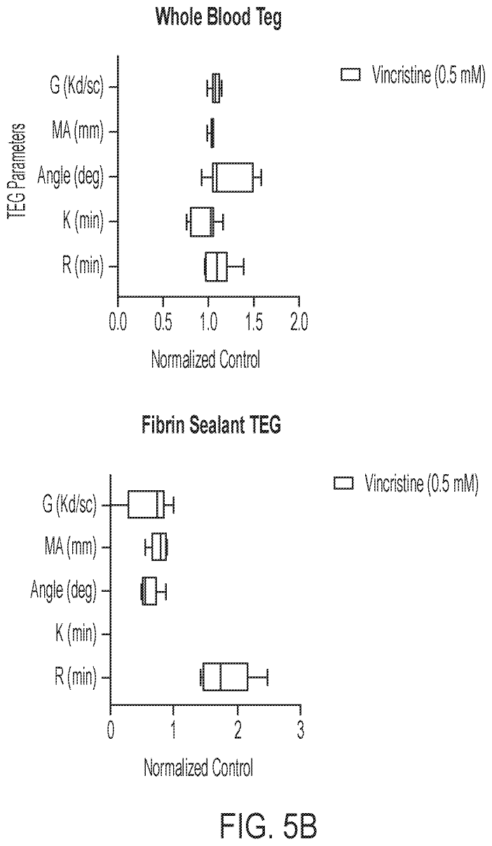

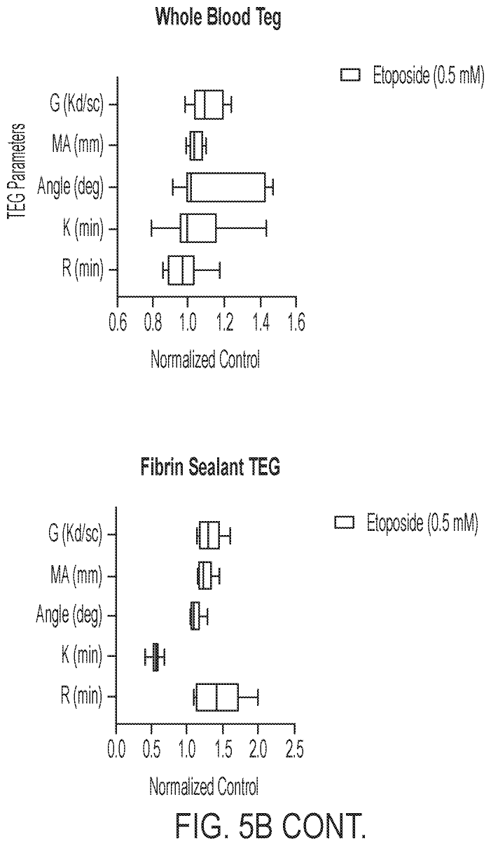

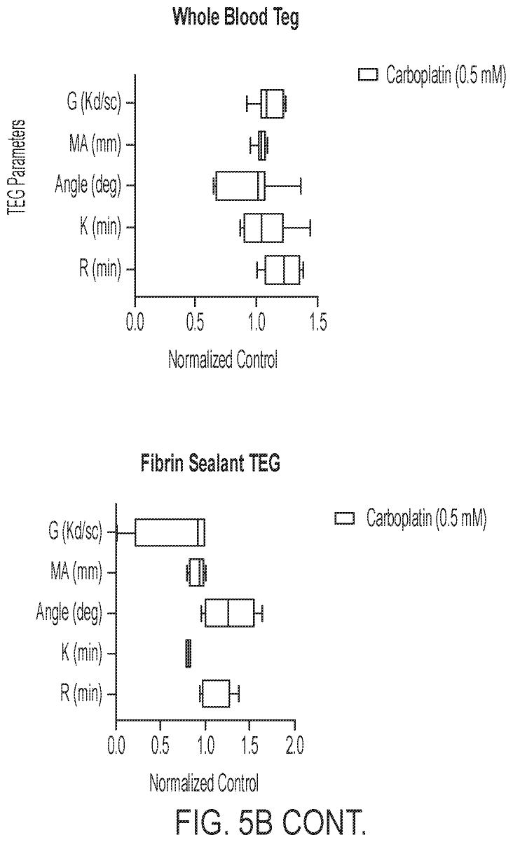

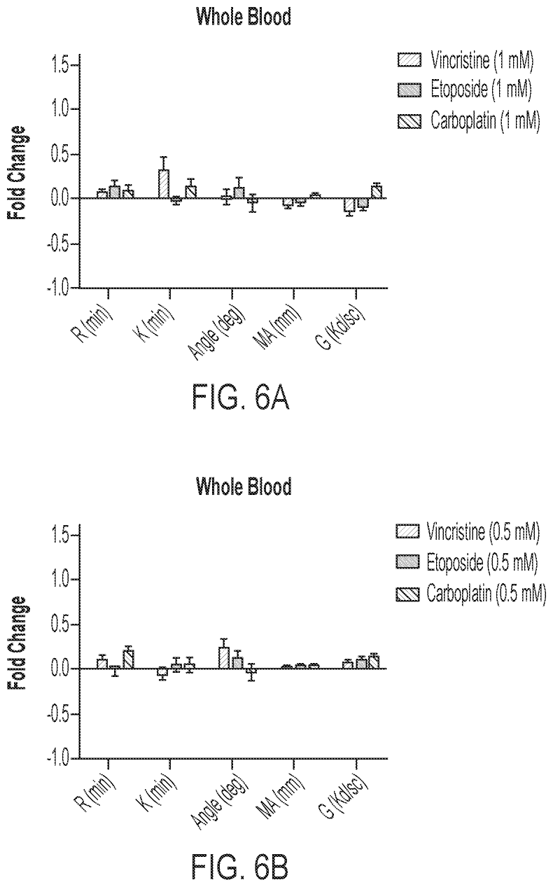

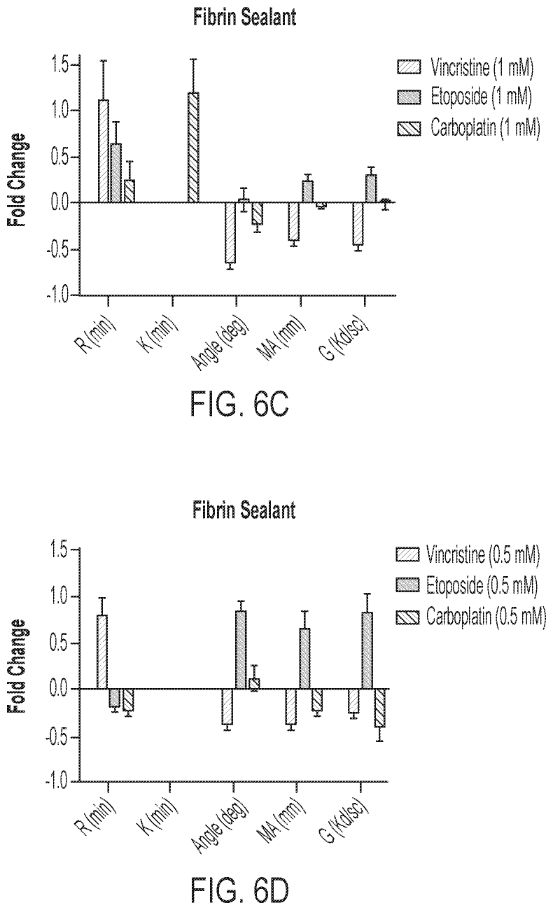

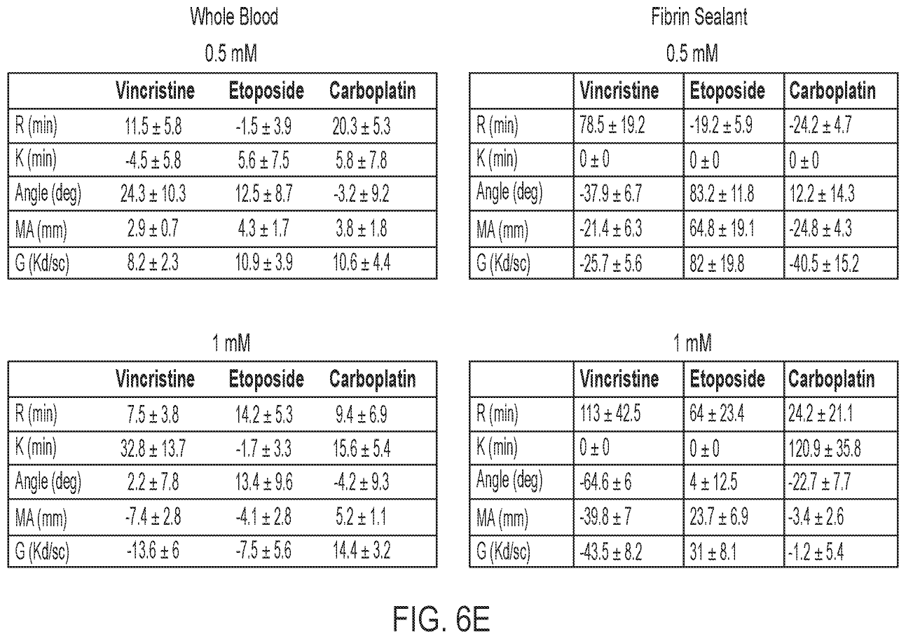

TEG Analytical Software version 4.2.3 was used to calculate the time to clot initiation time (R, minutes), time to clot firmness (K, minutes), alpha angle (a, degrees), maximal clot strength (MA, maximum amplitude, mm), and shear elastic modulus strength (G, dynes/cm²). Analyses were performed for at least 30 minutes or until the operating software had calculated all mentioned parameters. The results are shown in FIG. 5 and FIGS. 6A to 6E.

The effects of VEC on fibrin formation were also determined from adapted densitometry or thromboelastography (TEG) techniques. Fibrin monomer formation was monitored (A405 nm) in VEC-supplemented (0.5-1 mM) plasma or fibrinogen concentrate following clot generation by calcium or thrombin. Viscoelastic properties of clot formation were determined in VEC-supplemented whole blood or fibrinogen concentrate triggered by thrombin.

TEG Analytical Software version 4.2.3 was used to calculate the time to clot initiation (R, minutes), time to clot firmness (K, minutes), alpha angle (a, degrees), maximal clot strength (MA, maximum amplitude, mm), and shear elastic modulus strength (G, dynes/cm²).

Fibrinokinetics

Citrated pool blood plasma was obtained from Loyola blood bank. Chemotherapeutics Vincristine, Etoposide, and Carboplatin were reconstituted in PBS or small concentrations of DMSO. Fibrinokinetics were measured in microtiter plates using 175 microliter of plasma supplemented with 25 uL drug [1 mM-0.25 mM], and using a solution of 50 uL of purified thrombin [5 IU/mL]/CaCl₂ [0.025M] to a trigger clot generation. For the control well plates 25 uL of DMSO or PBS was added. The rate of clot formation was monitored for 40 minutes at A405 nm. Both kinetic and endpoint methods were used to calculate the rate of clot formation.

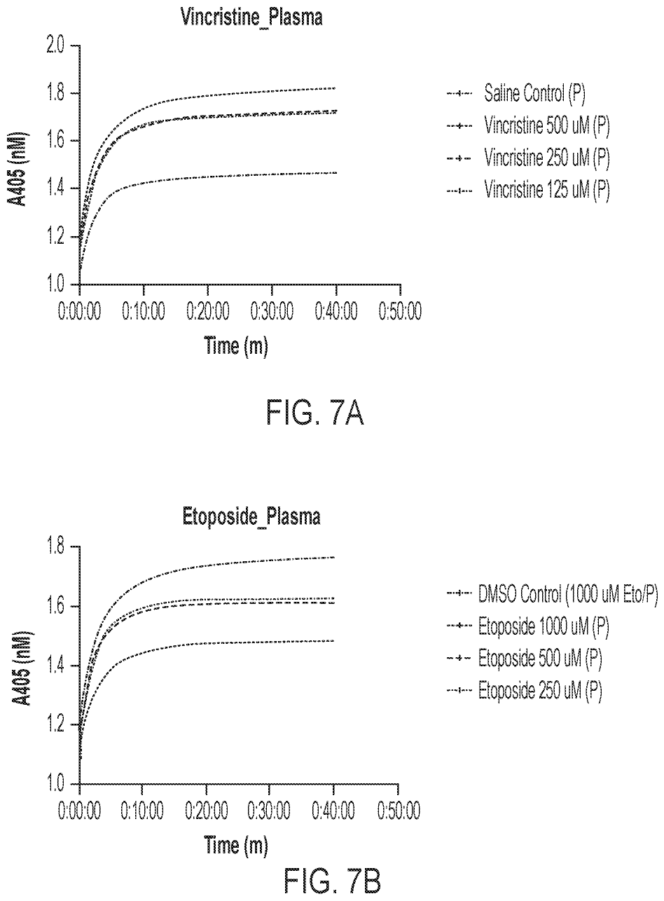

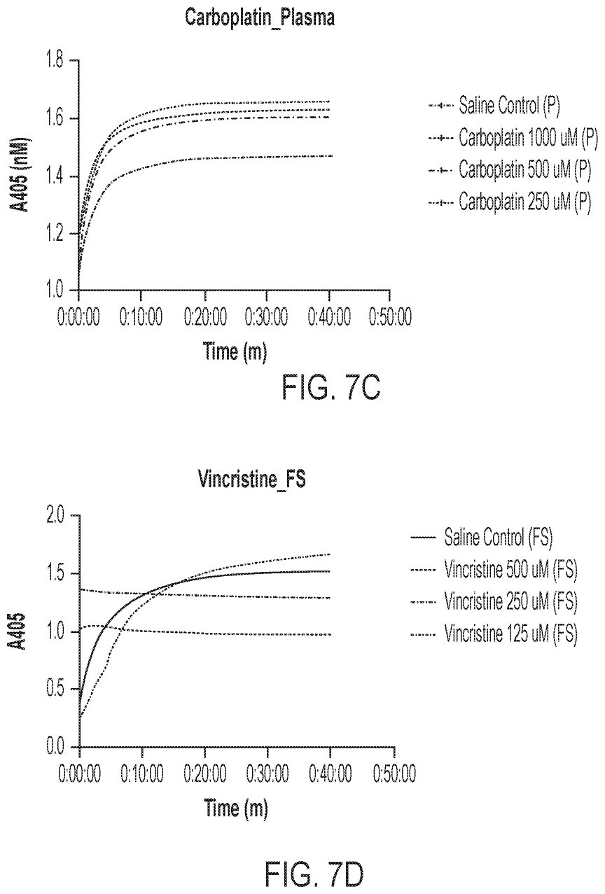

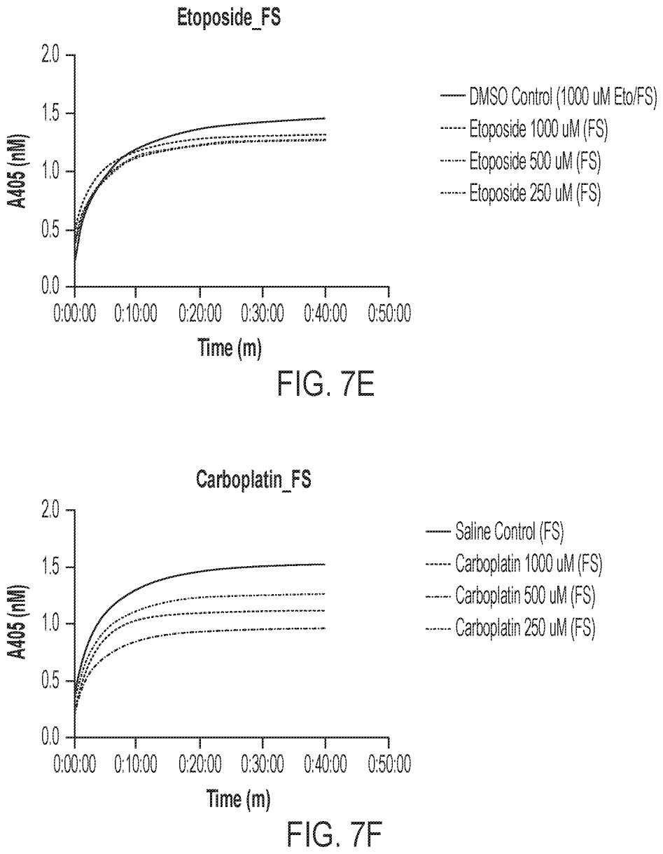

TISSEEL™ fibrinogen protein concentrate was diluted to physiological plasma concentrations (˜3 mg/mL). Chemotherapeutics Vincristine, Etoposide, and Carboplatin were reconstituted in PBS or small concentrations of DMSO. Fibrinokinetics were measured in microtiter plates using 175 microliter of plasma supplemented with 25 uL drug [1 mM-0.25 mM], and using a solution of 50 uL of purified thrombin [5 IU/mL]/CaCl₂ [0.025M] to a trigger clot generation. For the control well plates 25 uL of DMSO or PBS was added. The rate of clot formation was monitored for 40 minutes at A405 nm. Both kinetic and endpoint methods were used to calculate the rate of clot formation. The results are provided in FIG. 7.

Static Dialysis

Three fibrinogen and thrombin protein vials were reconstituted at 37 degrees. Following reconstitution, the thrombin solutions were diluted with reconstituted doxorubicin in water to bring the final thrombin concentration of three 0.5 mL samples containing 5 IU/mL, 100 IU/mL, or 500 IU/mL; each containing 7.5 mM Doxorubicin HCl (1.88 grams).

Following solution preparation, thrombin and fibrinogen solutions, each solution was pulled up into respective syringes and loaded in a Duploject spray system and sprayed into 300 uL PELCO 105 molds and allowed to coagulate for 1 minutes at room temperature. The molds were then placed in 5 cm×10 mm pre-soaked and rinsed Spectra/Por Biotech regenerated cellulose dialysis membrane tubing (Spectrum Laboratories, MWCO: 3.5-5 kDa), and the suspension dialyzed at 37° C. by immersion in 20 ml PBS (pH 7.4) containing 0.05% NaN₃ with continuous gentle stirring. At timed intervals, a 50 uL aliquot of dialysate was removed and replaced with an equal volume of PBS. Doxorubicin present in the dialysate was quantified by HPLC as described above, with an external standard curve prepared in water.

To determine the actual delivered drug concentrations, two molds were dissolved by 0.05% trypsin at 37 degrees Celsius, centrifuged at 21,000 g to remove most of the protein content, and the supernatant verified by HPLC.

Generating HPLC Standard Curves

Chemotherapy drugs were resolved by HPLC (Hitachi LaChrom Elite, model 2200) using a Beckman Coulter Ultrasphere 5 μm C18 reverse-phase column (4.6 mm×25 cm). An isocratic mobile phase of acetonitrile and formic acid (0.05N) was used to elute each respective drug from the column at a rate of 1 ml/min. Table 1 provides the mobile phase and detection parameters used for each drug standard created.

TABLE 1

| | % Formic | | | Retention |
|---|---|---|---|---|
| | Acid | % | Wavelength | Time |
| Drug | (0.05N) | Acetonitrile | Detection | (min) |
| Carboplatin | 98.7 | 1.3 | 230 | 4.39 |
| Carmustine | 30 | 70 | 230 | 3.39 |
| Doxorubicin | 70 | 30 | 233 | 4.92 |
| Etoposide | 30 | 70 | 280 | 3.37 |

Method development occurred following literature searches for HPLC parameters and instrumentation used for each drug. A variety of acetonitrile:formic acid ratios were attempted for each drug until a satisfactory peak was resolved. Max absorption wavelengths were determined using a NanoDrop2000 (ThermoFisher Scientific) and were used as UV-Vis detection wavelength. Standard curves were generated for each drug, by creating known drug standards between (0.1-1 ug/uL). Solutions were run in triplicate, the areas under the detected drug peaks integrated, and linear fits were generated.

Scanning Electron Microscopy

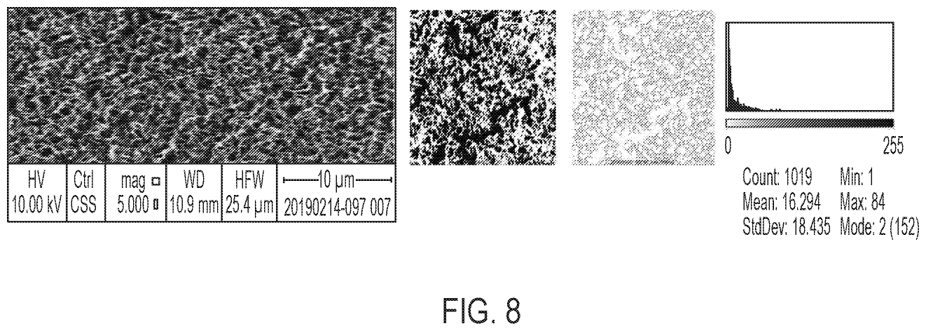

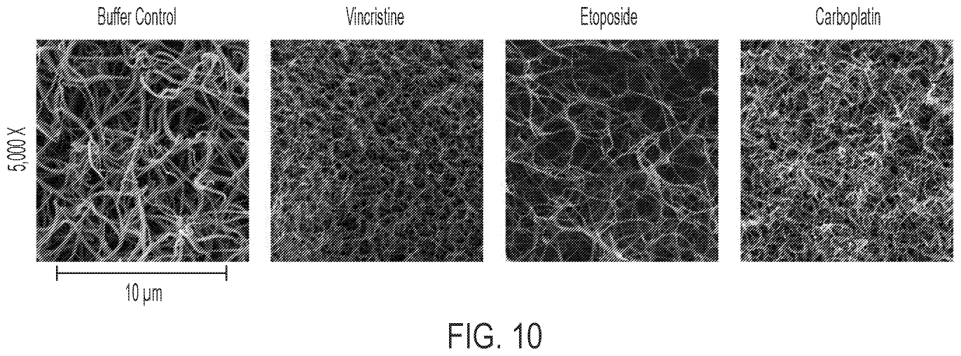

TISSEEL™ or TISSEEL™ admixed with chemotherapeutic drug (1 mM final concentration) was prepared according to the IFU. In short, 2 mM additions of chemotherapy were transferred via syringe and needle to the thrombin vial (500 IU/mL thrombin) during preparation and placed in a Fibrinotherm to spin and heat the mixtures to 37 degrees celsius. Following complete dissolution of both thrombin and fibrinogen solutions, each solution was pulled up into respective syringes and loaded in the Duploject spray system accompanying the kit. The 1 mL volumes of thrombin and fibrinogen (2 mL total) were sprayed onto collagen casing. Immediately following coagulation (˜30 seconds), the samples were washed 3× in cacodylate buffer for 10 minutes and fixed in 2% glutaraldehyde buffer (Electron Microscopy Sciences). Fixed clots were washed, serially dehydrated with ethanol, and dried with hexamethyldisilizane (Electron Microscopy Sciences). Clots were allowed to air dry overnight thereafter. Clots were then teased apart with forceps and mounted on aluminum sample supports with carbon adhesive tape. Samples were sputter coated with 20 nm iridium metal in a Leica ACE600 and analyzed by scanning electron microscopy (FEI Quanta 650 FEG). The results are provided in FIGS. 8 and 10.

Branching and Pore Size Analysis

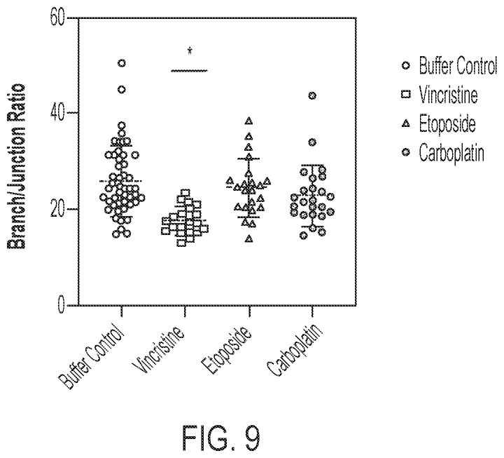

Branching analysis was performed using 2D images cross-sectional cuts of fibrin clots. Representative images and sections displaying easily discernible fibrin branching characteristics were selected from each group. Images were processed using ImageJ's skeleton analysis function. In short, images were first binarized to delineate the image further into branch and background sections. Binary images were then placed through the Analyze Skeleton (2D/3D) function with no pruning or selected parameters. The output file included details such as branch lengths, branch numbers, and number of junctions. The results are provided in FIG. 9 and Table 2.

TABLE 2

| | | | | Branch/ |
|---|---|---|---|---|
| | Max Branch | | Avg Branch | Junction |
| | Number | Junctions | length (nm) | Ratio |
| Control | 5043 +/− 108 | 71 +/− 1.5 | 26 | 71.0 |
| Vincristine | 7580 +/− 125 | 136 +/− 2.3 | 22 | 55.7 |
| Etoposide | 8272 +/− 140 | 125 +/− 2.1 | 21 | 66.2 |
| Carboplatin | 10065 +/− 142 | 148 +/− 2.6 | 19 | 68.0 |

### Example 2

### Results

Standalone IC50's calculated for Vincristine, Etoposide, or Carboplatin in WERI cells were 2.9 uM, 2.7 uM, 1.0 uM; or in Y79 cells were 0.1 uM, 6.0 uM, and 1.1 uM respectively. Fixed-ratio VEC 72-hour combination treatments in WERI cells suggest synergistic effects at sub-micromolar concentrations, but antagonistic half maximal concentrations for any drugs alone. Fibrin polymerization and viscoelastic clot properties were unaffected by incorporation of etoposide and carboplatin in both whole blood and sealant formulations. Alternatively, vincristine impeded fibrin formation time, but did not affect maximal clot amplitude or modulus. These results were recapitulated by EM network analysis, in which branch-to-junction ratios significantly were significantly lower in V-embedded fibrin matrices (55.7) compared to saline controls (71.0).

Combined treatments performed antagonistically at levels needed to effectively inhibit tumor growth (>90%). Deviations in fibrin clot formation characteristics (as seen with high-dose 1 mM vincristine supplementation) may play a role VEC release rates and clot breakdown in vivo.

What is claimed is:

1. A method for treating retinoblastoma comprising: administering a composition comprising two or more chemotherapeutic agents and a fibrin sealant material to a patient in need thereof.

2. The method of claim 1, wherein the chemotherapeutic agents are selected from the group consisting of: vincristine, etoposide, carboplatin, melphalan, doxorubicin, fluorouracil, carmustine, methotrexate, and mixtures thereof.

3. The method of claim 1, wherein the chemotherapeutic agents are a mixture of vincristine, etoposide, and carboplatin.

4. The method of claim 1, wherein each chemotherapeutic agent is independently present in the composition in a concentration of about 1 uM to about 1 mM, such as about 10 uM to about 1 mM, about 50 uM to about 500 uM, about 100 uM to about 250 uM, about 100 uM to about 200 uM, about 100 uM to about 150 uM, about 1 uM to about 5 uM, about 5 uM to about 10 uM, about 10 uM to about 20 uM, about 20 uM to about 30 uM, about 30 uM to about 40 uM, about 40 uM to about 50 uM, about 50 uM to about 60 uM, about 60 uM to about 70 uM, about 70 uM to about 80 uM, about 80 uM to about 90 uM, about 90 uM to about 100 uM, about 100 uM to about 120 uM, about 120 uM to about 140 uM, about 140 uM to about 160 uM, about 160 uM to about 180 uM, about 180 uM to about 200 uM, about 200 uM to about 220 uM, about 220 uM to about 240 uM, about 240 uM to about 260 uM, about 260 uM to about 280 uM, about 280 uM to about 300 uM, about 300 uM to about 350 uM, about 350 uM to about 400 uM, about 400 uM to about 500 uM, about 500 uM to about 600 uM, about 600 uM to about 700 uM, about 700 uM to about 800 uM, about 800 uM to about 900 uM, and/or about 900 uM to about 1 mM.

5. The method of claim 1, wherein the chemotherapeutic agents are present in the composition in a total concentration of about 3 um to about 3 mM, such as about 30 uM to about 3 mM, about 150 uM to about 1.5 mM, about 300 uM to about 750 uM, about 300 uM to about 600 uM, about 300 uM to about 450 uM, about 3 uM to about 15 uM, about 15 uM to about 30 uM, about 30 uM to about 60 uM, about 60 uM to about 90 uM, about 90 uM to about 120 uM, about 120 uM to about 150 uM, about 150 uM to about 180 uM, about 180 uM to about 210 uM, about 210 uM to about 240 uM, about 240 uM to about 270 uM, about 270 uM to about 300 uM, about 300 uM to about 360 uM, about 360 uM to about 420 uM, about 420 uM to about 480 uM, about 480 uM to about 540 uM, about 540 uM to about 600 uM, about 600 uM to about 660 uM, about 660 uM to about 720 uM, about 720 uM to about 780 uM, about 780 uM to about 840 uM, about 840 uM to about 900 uM, about 900 uM to about 1050 uM, about 1050 uM to about 1200 uM, about 1200 uM to about 1.5 mM, about 1.5 mM to about 1.8 mM, about 1.8 mM to about 2.1 mM, about 2.1 mM to about 2.4 mM, about 2.4 mM to about 2.7 mM, and/or about 2.7 to about 3 mM.

6. The method of claim 1, wherein the chemotherapeutic agents are a mixture of vincristine, etoposide, and carboplatin, and the vincristine is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%; or the vincristine is present in an amount of about 0.05% to about 10% of the chemotherapeutical agents on a molar basis, such as about 0.05% to about 0.1%, about 0.1% to about 0.2%, about 0.2% to about 0.3%, about 0.3% to about 0.4%, about 0.4% to about 0.5%, about 0.5% to about 1%, about 1% to about 2%, about 2% to about 5%, and/or about 5% to about 10%.

7. The method of claim 1, wherein the chemotherapeutic agents are a mixture of vincristine, etoposide, and carboplatin, and the etoposide is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

8. The method of claim 1, wherein the chemotherapeutic agents are a mixture of vincristine, etoposide, and carboplatin, and the carboplatin is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

9. The method of claim 1, wherein the chemotherapeutic agents are a mixture of vincristine, etoposide, and carboplatin, and the chemotherapeutic agents are present in an amount of about 0.05% to about 10% vincristine, about 10% to about 80% etoposide, and about 10% to about 80% carboplatin on a molar basis of the chemotherapeutical agents, such as about 0.1% to about 5% vincristine, about 15% to about 60% etoposide, and about 40% to about 80% carboplatin, about 0.1% to about 1% vincristine, about 20% to about 50% etoposide, and about 50% to about 80% carboplatin, about 0.1% to about 0.5% vincristine, about 25% to about 45% etoposide, and about 55% to about 75% carboplatin, about 0.1% to about 0.2% vincristine, about 30% to about 40% etoposide, and about 60% to about 70% carboplatin, and/or about 0.2% vincristine, about 34.9% etoposide, and about 64.9% carboplatin.

10. The method of claim 1, wherein the fibrin sealant material comprises fibrinogen and thrombin.

11. The method of claim 1, wherein the fibrin sealant material comprises fibrinogen in a concentration of about 10 mg/mL to about 200 mg/mL, such as about 30 mg/mL to about 150 mg/mL, about 50 mg/mL to about 120 mg/mL, about 75 mg/mL to about 125 mg/mL, about 80 mg/mL to about 100 mg/mL, about 90 mg/mL, about 10 mg/mL to about 30 mg/mL, about 30 mg/mL to about 50 mg/mL, about 50 mg/mL to about 70 mg/mL, about 70 mg/mL to about 90 mg/mL, about 90 mg/mL to about 110 mg/mL, about 110 mg/mL to about 130 mg/mL, about 130 mg/mL to about 150 mg/mL, about 150 mg/mL to about 170 mg/mL, and/or about 170 mg/mL to about 200 mg/mL.

12. The method of claim 1, wherein the fibrin sealant material comprises thrombin in a concentration of about 10 IU/mL to about 2000 IU/mL, such as about 50 IU/mL to about 1000 IU/mL, about 100 IU/mL to about 800 IU/mL, about 250 IU/mL to about 750 IU/mL, about 300 IU/mL to about 700 IU/mL, about 400 IU/mL to about 600 IU/mL, and/or about 500 IU/mL.

13. The method of claim 1 wherein the composition is administered using a dual-compartment syringe.

14. The method of claim 13, wherein a first compartment of the syringe comprises the chemotherapeutic agents and thrombin, and a second compartment of the syringe comprises fibrinogen.

15. The method of claim 13, wherein a first compartment of the syringe comprises the chemotherapeutic agents and fibrinogen, and a second compartment of the syringe comprises thrombin.

16. The method of claim 1, wherein the composition is administered by subconjunctival injection.

17. A composition for treating retinoblastoma comprising: two or more chemotherapeutic agents and a fibrin sealant material.

18. The composition of claim 17, wherein the chemotherapeutic agents are selected from the group consisting of: vincristine, etoposide, carboplatin, melphalan, doxorubicin, fluorouracil, carmustine, methotrexate, and mixtures thereof.

19. The composition of claim 17, wherein the chemotherapeutic agents are a mixture of vincristine, etoposide, and carboplatin.

20. The composition of claim 17, wherein each chemotherapeutic agent is independently present in the composition in a concentration of about 1 uM to about 1 mM, such as about 10 uM to about 1 mM, about 50 uM to about 500 uM, about 100 uM to about 250 uM, about 100 uM to about 200 uM, about 100 uM to about 150 uM, about 1 uM to about 5 uM, about 5 uM to about 10 uM, about 10 uM to about 20 uM, about 20 uM to about 30 uM, about 30 uM to about 40 uM, about 40 uM to about 50 uM, about 50 uM to about 60 uM, about 60 uM to about 70 uM, about 70 uM to about 80 uM, about 80 uM to about 90 uM, about 90 uM to about 100 uM, about 100 uM to about 120 uM, about 120 uM to about 140 uM, about 140 uM to about 160 uM, about 160 uM to about 180 uM, about 180 uM to about 200 uM, about 200 uM to about 220 uM, about 220 uM to about 240 uM, about 240 uM to about 260 uM, about 260 uM to about 280 uM, about 280 uM to about 300 uM, about 300 uM to about 350 uM, about 350 uM to about 400 uM, about 400 uM to about 500 uM, about 500 uM to about 600 uM, about 600 uM to about 700 uM, about 700 uM to about 800 uM, about 800 uM to about 900 uM, and/or about 900 uM to about 1 mM.

21. The composition of claim 17, wherein the chemotherapeutic agents are present in the composition in a total concentration of about 3 um to about 3 mM, such as about 30 uM to about 3 mM, about 150 uM to about 1.5 mM, about 300 uM to about 750 uM, about 300 uM to about 600 uM, about 300 uM to about 450 uM, about 3 uM to about 15 uM, about 15 uM to about 30 uM, about 30 uM to about 60 uM, about 60 uM to about 90 uM, about 90 uM to about 120 uM, about 120 uM to about 150 uM, about 150 uM to about 180 uM, about 180 uM to about 210 uM, about 210 uM to about 240 uM, about 240 uM to about 270 uM, about 270 uM to about 300 uM, about 300 uM to about 360 uM, about 360 uM to about 420 uM, about 420 uM to about 480 uM, about 480 uM to about 540 uM, about 540 uM to about 600 uM, about 600 uM to about 660 uM, about 660 uM to about 720 uM, about 720 uM to about 780 uM, about 780 uM to about 840 uM, about 840 uM to about 900 uM, about 900 uM to about 1050 uM, about 1050 uM to about 1200 uM, about 1200 uM to about 1.5 mM, about 1.5 mM to about 1.8 mM, about 1.8 mM to about 2.1 mM, about 2.1 mM to about 2.4 mM, about 2.4 mM to about 2.7 mM, and/or about 2.7 to about 3 mM.

22. The composition of claim 17, wherein the chemotherapeutic agents are a mixture of vincristine, etoposide, and carboplatin, and the vincristine is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%; or the vincristine is present in an amount of about 0.05% to about 10% of the chemotherapeutical agents on a molar basis, such as about 0.05% to about 0.1%, about 0.1% to about 0.2%, about 0.2% to about 0.3%, about 0.3% to about 0.4%, about 0.4% to about 0.5%, about 0.5% to about 1%, about 1% to about 2%, about 2% to about 5%, and/or about 5% to about 10%.

23. The composition of claim 17, wherein the chemotherapeutic agents are a mixture of vincristine, etoposide, and carboplatin, and the etoposide is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

24. The composition of claim 17, wherein the chemotherapeutic agents are a mixture of vincristine, etoposide, and carboplatin, and the carboplatin is present in an amount of about 10% to about 90% of the chemotherapeutical agents on a molar basis, such as about 10% to about 20%, about 20% to about 30%, about 30% to about 40%, about 40% to about 50%, about 50% to about 60%, about 60% to about 70%, about 70% to about 80%, and/or about 80% to about 90%.

25. The composition of claim 17, wherein the chemotherapeutic agents are a mixture of vincristine, etoposide, and carboplatin, and the chemotherapeutic agents are present in an amount of about 0.05% to about 10% vincristine, about 10% to about 80% etoposide, and about 10% to about 80% carboplatin on a molar basis of the chemotherapeutical agents, such as about 0.1% to about 5% vincristine, about 15% to about 60% etoposide, and about 40% to about 80% carboplatin, about 0.1% to about 1% vincristine, about 20% to about 50% etoposide, and about 50% to about 80% carboplatin, about 0.1% to about 0.5% vincristine, about 25% to about 45% etoposide, and about 55% to about 75% carboplatin, about 0.1% to about 0.2% vincristine, about 30% to about 40% etoposide, and about 60% to about 70% carboplatin, and/or about 0.2% vincristine, about 34.9% etoposide, and about 64.9% carboplatin.

26. The composition of claim 17, wherein the fibrin sealant material comprises fibrinogen and thrombin.

27. The composition of claim 17, wherein the fibrin sealant material comprises fibrinogen in a concentration of about 10 mg/mL to about 200 mg/mL, such as about 30 mg/mL to about 150 mg/mL, about 50 mg/mL to about 120 mg/mL, about 75 mg/mL to about 125 mg/mL, about 80 mg/mL to about 100 mg/mL, about 90 mg/mL, about 10 mg/mL to about 30 mg/mL, about 30 mg/mL to about 50 mg/mL, about 50 mg/mL to about 70 mg/mL, about 70 mg/mL to about 90 mg/mL, about 90 mg/mL to about 110 mg/mL, about 110 mg/mL to about 130 mg/mL, about 130 mg/mL to about 150 mg/mL, about 150 mg/mL to about 170 mg/mL, and/or about 170 mg/mL to about 200 mg/mL.

28. The composition of claim 17, wherein the fibrin sealant material comprises thrombin in a concentration of about 10 IU/mL to about 2000 IU/mL, such as about 50 IU/mL to about 1000 IU/mL, about 100 IU/mL to about 800 IU/mL, about 250 IU/mL to about 750 IU/mL, about 300 IU/mL to about 700 IU/mL, about 400 IU/mL to about 600 IU/mL, and/or about 500 IU/mL.

29. The composition of claim 17, wherein the fibrin sealant material is obtained by combining a first solution comprising the chemotherapeutical agents and thrombin with a second solution comprising fibrinogen.

30. The composition of claim 17, wherein the fibrin sealant material is obtained by combining a first solution comprising fibrinogen with a second solution comprising the chemotherapeutical agents and thrombin.

31. A dual-compartment syringe comprising two or more chemotherapeutic agents, fibrinogen, and thrombin.

32. The dual-compartment syringe of claim 31, wherein a first compartment of the syringe comprises the chemotherapeutic agents and thrombin, and a second compartment of the syringe comprises fibrinogen.

33. The dual-compartment syringe of claim 32, wherein the chemotherapeutic agents and thrombin are present as a solution, and the fibrinogen is present as a solution.

34. The dual-compartment syringe of claim 33, wherein the solutions are frozen.

35. The dual-compartment syringe of claim 32, wherein the chemotherapeutic agents and thrombin are present as a dry powder, and the fibrinogen is present as a dry powder.

36. The dual-compartment syringe of claim 35, wherein the dry powders are reconstituted to form a solution.

37. The dual-compartment syringe of claim 31, wherein a first compartment of the syringe comprises the chemotherapeutic agents and fibrinogen, and a second compartment of the syringe comprises thrombin.

38. The dual-compartment syringe of claim 37, wherein the chemotherapeutic agents and thrombin are present as a solution, and the fibrinogen is present as a solution.

39. The dual-compartment syringe of claim 38, wherein the solutions are frozen.

40. The dual-compartment syringe of claim 37, wherein the chemotherapeutic agents and thrombin are present as a dry powder, and the fibrinogen is present as a dry powder.

41. The dual-compartment syringe of claim 40, wherein the dry powders are reconstituted to form a solution.
